(19) United States
(12) Patent Application Publication
(10) Pub. No.: US 2005/0169976 A1
(43) Pub. Date: Aug. 4, 2005

(54) Insulin administration apparatus

(76) Inventors: Kenji Mori, Ibaraki (JP); Naruhito Higo, Ibaraki (JP); Seiji Tokumoto, Ibaraki (JP); Shuji Sato, Kanagawa (JP); Kenji Sugibayashi, Saitama (JP)

Correspondence Address: TOWNSEND & BANTA;c/o PORTFOLIO IP, PO BOX 52050, MINNEAPOLIS, MN, 55402, US

(21) Appl. No.: 10/510,694

(22) PCT Filed: Mar. 28, 2003
(86) PCT No.: PCT/JP03/03992

(30) Foreign Application Priority Data
Apr. 8, 2002 (JP) 2002-105796

(51) Int. Cl. A61N 1/30; A61K 38/28

(52) U.S. Cl. 424/449; 604/20; 514/3

(57) ABSTRACT

The present invention provides an insulin-administering device enabling the effective administration of insulin via a percutaneous or submucous administration route.
The present device is used to percutaneously or transmucosally administer an insulin lispro represented by the structural formula indicated below, or a pharmaceutically acceptable salt thereof, using iontophoresis and electroporation.

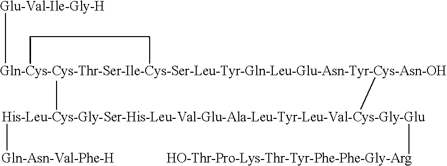

### TECHNICAL FIELD

The present invention relates to an insulin-administering device for percutaneously or transmucosally administering a lispro, using power of electric fields.

### BACKGROUND ART

Patients with diabetes are broadly classified into patients with type II diabetes, who can be treated with oral antidiabetics such as sulfonylurea, and patients with type I diabetes, who secrete no insulin. Since patients with type I diabetes secrete no insulin, they require administration of insulin. Even in the case of patients with type II diabetes, when it is difficult for them to control blood glucose, a therapeutic method for administering insulin is applied, as in the case of patients with type I diabetes. However, while insulin has high blood glucose-controlling effects, it is poor in absorptivity and stability. Thus, insulin cannot be orally administered. In addition, insulin has low sustainability. Under the current circumstances, insulin is administered by injection on a frequent basis from one to several times a day. However, such injection involves certain amount of pain. International Publication WO02/02179A1 describes an example of percutaneously administering insulin or insulin lispro, using a microneedle. Such amicroneedle causes only a small degree of pain. However, such a microneedle physically creates a very small pore on the skin so as to percutaneously inject a drug though the pore, and the pore remains even after administration. Thus, regarding the use of a microneedle, problems such as infectious diseases are not negligible.

In addition, as methods for promoting the absorption of drugs through the skin or mucosa, administration methods using electric energy, such as iontophoresis (Journal of Pharmaceutical Sciences, Vol. 76, p. 341, 1987) and electroporation (National Publication of International Patent Application No. 1991-502416; Proc. Natl. Acad. Sci. U.S.A., Vol. 90, pp. 10504-10508, 1993), have been developed. Since iontophoresis delivers drugs through hair follicles located on the skin, a pore or the like is not generated on the skin. In addition, since the applied voltage is low, this method is extremely safe. Electroporation involves the application of high voltage, but the application time is very short, such as a period ranging from several microseconds to several milliseconds. A pore generated as a result of electroporation is reversible, and thus, it does not remain after completion of the administration of drugs. Both iontophoresis and electroporation are safe administration methods, which promote the absorption of drugs percutaneously or transmucosally.

Nevertheless, it is difficult to administer insulin even using these techniques. For example, there has been a report regarding the delivery of insulin by a single use of iontophoresis (Journal of Pharmaceutical Sciences, Vol. 76, p. 341, 1987). However, almost no effects were obtained by this method. Although a decrease in the blood glucose level was observed, such effects lasted just over ten minutes. In addition, there have been no reports that a sufficient amount of insulin was delivered by the use of electroporation to such an extent that the effects of the insulin delivered were observed. Moreover, although the combined use of electroporation and iontophoresis has also been studied, there have been no reports that a sufficient amount of insulin was delivered by such a combined use to such an extent that the effects of the insulin delivered were observed. Regarding an attempt to cause drugs other than insulin to be percutaneously absorbed by the combined use of electroporation and iontophoresis, there has been a report that calcitonin with a molecular weight of 3000 could be delivered to such an extent that a rat had a blood calcitonin level of a few hundred ng/mL, but that PTH with a molecular weight of 4000 could be delivered only in small amounts that were less than 100 ng/mL (Journal of Controlled Release, Vol. 66, p. 127, 2000). This is to say, under the current circumstances, it is difficult to deliver compounds with a molecular weight of more than 3000 by the combined use of electroporation and iontophoresis. Furthermore, it is also difficult to deliver a sufficient amount of insulin, which has a molecular weight of 6000, through the skin or mucosa.

Accordingly, it is an object of the present invention to provide an insulin-administering device enabling the effective administration of insulin via a percutaneous or submucous administration route.

### DISCLOSURE OF THE INVENTION

In order to achieve the aforementioned object, the present inventor has used iontophoresis capable of applying a low electric field for a long time and electroporation capable of applying a high electric field for a short time, singly or in combination. Thus, the present inventor has attempted to administer various types of insulin (human insulin, swine insulin, bovine insulin, arginine-insulin, and insulin lispro).

As a result, the present inventor has found that when insulin lispro represented by the structural formula indicated below or a pharmaceutically acceptable salt thereof (hereinafter referred to as “insulin lispro”) is used, electroporation is combined with iontophoresis as means for applying electric fields, so that excellent percutaneous or submucous absorptivity of the drug can be achieved, and that the above drug can exhibit sufficient beneficial effects and maintain such effects for a long time, thereby completing the present invention.

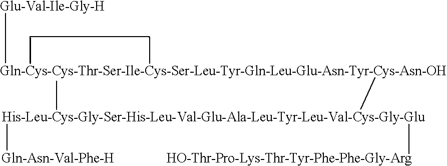

That is to say, the insulin-administering device of the present invention percutaneously or transmucosally administers the above insulin lispro, using at least two different electric field-applying means. Herein, the two different electric field-applying means are preferably iontophoresis and electroporation. In this case, the electric current applied during iontophoresis is preferably between 0.01 and 1.0 mA/cm². In addition, the voltage applied during electroporation is preferably between 1 V/cm and 10 kV/cm per unit distance between electrodes.

Moreover, said insulin lispro is preferably dissolved, suspended, or dispersed in a hydrophilic matrix. Such a hydrophilic matrix may comprise one or more selected from the group consisting of agar, locust bean gum, xanthan gum, polyvinyl alcohols and derivatives thereof, and polyacrylic acid and salts thereof.

Furthermore, the insulin-administering device of the present invention may comprise a membrane for controlling the release of an insulin lispro. At least a pair of electrodes used for electroporation is disposed on the release-controlling membrane. The release-controlling membrane is preferably formed from a porous membrane. An insulin lispro may be retained on the membrane. In this case, it is preferable that an insulin lispro be retained in a dry state on the membrane and that a part or all of said insulin lispro be dissolved when they are used. At least one of the electrodes used for electroporation is preferably disposed directly on the skin or mucosa, or adjacent thereto (for example, at a distance of approximately 100 μm or less).

Still further, the insulin-administering device of the present invention comprises an electroporation-iontophoresis formulation containing the above insulin lispro, a reference formulation that is a counter electrode in iontophoresis, and a power supply connected to both formulations. Herein, the power supply has a connecting port used for iontophoresis and a connecting port used for electroporation.

Still further, the electroporation-iontophoresis formulation of the present invention comprises a backing, aniontophoresis electrode disposed on the backing, an insulin lispro-containing layer which is disposed on the iontophoresis electrode and contains the above insulin lispro, and electroporation electrodes which are disposed on the insulin lispro-containing layer and have polarities different from each other. Herein, a release-controlling membrane for controlling the release of insulin lispro may be provided between the insulin lispro-containing layer and the electroporation electrodes. The release-controlling membrane may be a porous membrane having a pore size between 0.01 and 10 μm.

Still further, the electroporation-iontophoresis formulation of the present invention comprises a backing, an iontophoresis electrode disposed on the backing, a hydrophilic matrix base disposed on the iontophoresis electrode, a liner disposed on the hydrophilic matrix base, a retaining membrane which is disposed on the liner and retains the above insulin lispro, and electroporation electrodes which are disposed on the retaining membrane and have polarities different from each other. Herein, said insulin lispro is preferably retained in a dry state on the retaining membrane. The electroporation electrodes may be formed as a multipoint contact-type.

According to the above described structure, an insulin-administering device enabling the effective administration of insulin via a percutaneous or submucous administration route can be obtained.

### BRIEF DESCRIPTION OF THE DRAWINGS

FIG. 1 is a view showing an embodiment of the insulin-administering device of the present invention;

FIG. 2 is a view showing an example of the electroporation-iontophoresis formulation of the present invention. FIG. (a) shows a sectional view thereof, and FIG. (b) shows a plan view thereof;

FIG. 3 is a view showing another example of the electroporation-iontophoresis formulation of the present invention. FIG. (a) shows a sectional view thereof, and FIG. (b) shows a plan view thereof;

FIG. 4 is a view showing another example of the electroporation-iontophoresis formulation of the present invention. FIG. (a) shows a sectional view thereof, and FIG. (b) shows a plan view thereof;

FIG. 5 is a view showing another example of the electroporation-iontophoresis formulation of the present invention. FIG. (a) shows a plan view of an applied plane thereof, FIG. (b) shows a plan view of a conductive layer thereof, FIG. (c) is a partial sectional view of a porous membrane thereof, and FIG. (d) is an overall sectional view thereof;

FIG. 6 is a view showing the electroporation-iontophoresis formulation used in the present examples. FIG. (a) is a perspective view thereof, FIG. (b) is a sectional view thereof, and FIG. (c) is a plan view thereof;

FIG. 7 is a graph showing the level of insulin lispro in the blood in Example 1 and Comparative Examples 1 and 2;

FIG. 8 is a graph showing a change in the level of glucose in the blood in Example 1 and Comparative Examples 1 and 2, as a ratio of the blood glucose level after administration to the blood glucose level at the initial stage (before administration);

FIG. 9 is a graph showing the level of insulin in the blood in Example 1 and Comparative Example 3;

FIG. 10 is a graph showing a change in the level of glucose in the blood in Example 1 and Comparative Example 3, as a ratio of the blood glucose level after administration to the blood glucose level at the initial stage (before administration);

FIG. 11 is a graph showing the level of insulin in the blood in Example 2 and Comparative Example 4; and

FIG. 12 is a graph showing a change in the level of glucose in the blood in Example 2 and Comparative Examples 4, 5, 6, and 7, as a ratio of the blood glucose level after administration to the blood glucose level at the initial stage (before administration);

### BEST MODE FOR CARRYING OUT THE INVENTION

The present invention will be described in detail below.

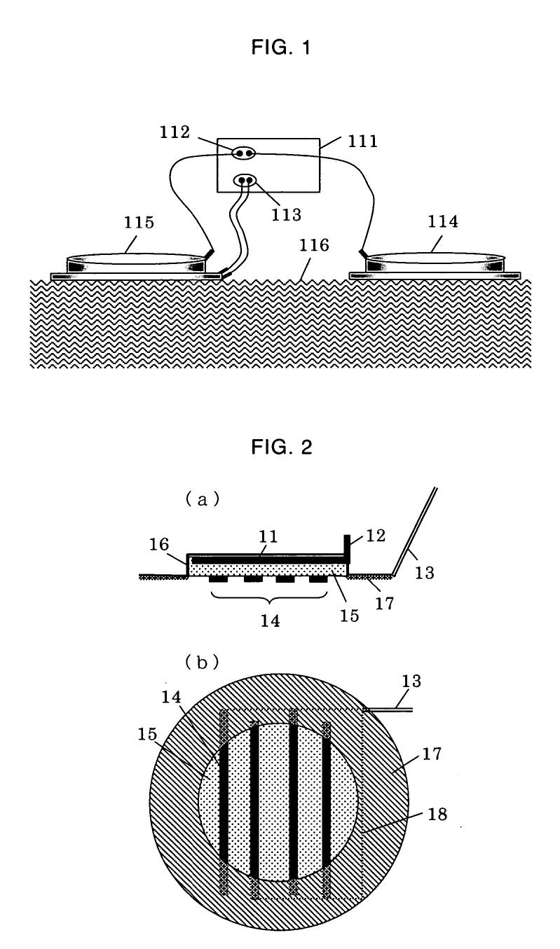

FIG. 1 is a view showing an embodiment of the insulin-administering device of the present invention. The present device includes iontophoresis and electroporation as two different electric field-applying means. As shown in FIG. 1, the present device comprises an electroporation-iontophoresis formulation 115 containing insulin lispro, a reference formulation 114 that is a counter electrode in iontophoresis, and an electroporation-iontophoresis power supply 111 which is connected to both formulations 114 and 115. The power supply 111 has an iontophoresis connecting port 112 and an electroporation connecting port 113. In the present example, both formulations 114 and 115 are applied to the skin 116.

In the present device, the power supply 111 allows the application of a high electric field during electroporation and also allows the application of a low electric field during iontophoresis. In this case, the voltage applied during electroporation is preferably between 1 V/cm and 10 kV/cm. In addition, the electric current applied during iontophoresis is preferably between 0.01 and 1.0 mA/cm² in terms of the amount of insulin delivered and electrical stimulation. Examples of a current waveform during iontophoresis may include a direct current, a pulse, and a pulse depolarization, but are not limited thereto. In constant current application in which the current value is constant, the electric current is preferably between 0.01 and 1.0 mA/cm², as stated above. In constant voltage application in which the voltage value is constant, the voltage is preferably between 1 V and 20V. When insulin lispro exists in an environment where pH is lower than its isoelectric point (around approximately 5.5), it is contained on the anode side of electrodes for iontophoresis. In contrast, when insulin lispro exists in an environment where pH is higher than its isoelectric point, it is contained on the cathode side thereof. Otherwise, insulin lispro may be contained on both the anode and cathode sides, so that it may be simultaneously administered from both sides. In this case, electroporation electrodes should also be disposed on both the anode and cathode formulations in iontophoresis.

FIG. 2 is a view showing an example of the electroporation-iontophoresis formulation of the present invention. FIG.(a) shows a sectional view thereof, and FIG.(b) shows a plan view thereof. As shown in the figures, the present formulation comprises: a backing 16 having a concave portion; an iontophoresis electrode 11 disposed on the bottom of the concave portion of the backing 16; an iontophoresis electrode-connecting terminal 12 for connecting the iontophoresis electrode 11 with an external power supply; an insulin lispro-containing layer 15 disposed inside the backing 16; an electroporation electrode 14 consisting of electrodes having different polarities from one another, which adjoin on a single plain surface disposed on the insulin lispro-containing layer 15; an electroporation electrode-connecting terminal 13 for connecting the electroporation electrode 14 with an external power supply; a conductor 18 for connecting the electroporation electrode 14 with the electroporation electrode-connecting terminal 13; and an adhesive insulator layer 17, which is attached to the skin to insulate such that the conductor 18 or the electroporation electrode 14 does not unnecessarily come into contact with the skin.

The insulin lispro-containing layer 15 comprises, as an active ingredient, one or more types of the above described insulin lispro. Pharmaceutically acceptable salts of such insulin lispro is not particularly limited, but generally used salts can be used herein.

Also, the insulin lispro-containing layer 15 preferably comprises a hydrophilic base capable of being dissolved, suspended, or dispersed in a matrix. Examples of such a base may include, but are not limited to, agar, gelatin, polyacrylic acid or a salt thereof, polyvinylpyrrolidone, a copolymer consisting of polyvinylpyrrolidone and vinyl acetate, methylcellulose or a derivative thereof, pectin, polyethylene oxide, a methyl vinyl ether-maleic anhydride copolymer, polyvinyl alcohol or a derivative thereof, and their saponified products.

Preferred examples of a material for an iontophoresis electrode may include silver and copper for the anode side, and non-polarized electrodes such as silver/silver chloride or copper/copper chloride for the cathode side. In addition, polarized electrodes such as carbon, titanium, gold, or platinum may also be used. Otherwise, non-polarized electrodes and polarized electrodes may be used in combination Any material may be used as a material for an electroporation electrode, as long as it causes electric current to pass through the material. Examples of such a material may include, but are not limited to, carbon, platinum, gold, silver, titanium, aluminum, chrome, zinc, and alloys thereof. Differing from the case of the iontophoresis electrode, a distance between the anode and the cathode is important for the electroporation electrode. This is because an electric field to be applied varies depending on such a distance. The distance is preferably within the range between 0.01 mm and 10 cm. It must be determined, considering voltage to be applied. For example, when a voltage of 10 V is applied with a distance of 10 cm between both electrodes, an electric field of 1 V/cm can be obtained. When a voltage of 1 V is applied with a distance of 0.01 mm between both electrodes, an electric field of 1000 V/cm can be obtained. A preferred applied electric field is between 1 V/cm and 10 kV/cm in the case of electroporation. The electroporation electrodes and the iontophoresis electrode may be shared, or may be established separately.

The iontophoresis reference formulation may have a structure used in common iontophoresis devices, although it is not shown in the figure. For example, the present formulation may have a structure formed by eliminating the electroporation electrode 14, the electroporation electrode-connecting terminal 13, and the conductor 18 from the structure shown in FIG. 2. In this case, the insulin lispro-containing layer 15 may be substituted by a simple conductive layer, which does not contain insulin lispro.

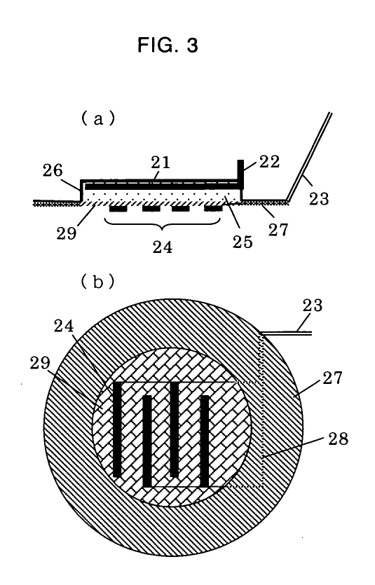

FIG. 3 is a view showing another example of the electroporation-iontophoresis formulation of the present invention. FIG. (a) shows a sectional view thereof, and FIG. (b) shows a plan view thereof. This formulation differs from the formulation shown in FIG. 2 in that it has a membrane for controlling the release of insulin lispro. This is to say, as shown in the figure, the present formulation comprises: a backing 26 having a concave portion; an iontophoresis electrode 21 disposed on the bottom of the concave portion of the backing 26; an iontophoresis electrode-connecting terminal 22 for connecting the iontophoresis electrode 21 with an external power supply; an insulin lispro-containing layer 25 disposed inside the backing 26; an insulin lispro release-controlling membrane 29 disposed on the insulin lispro-containing layer 25; an electroporation electrode 24 consisting of electrodes having different polarities from one another, which are fixed or printed on the insulin lispro release-controlling membrane 29, and adjoin on a single plain surface; an electroporation electrode-connecting terminal 23 for connecting the electroporation electrode 24 with an external power supply; a conductor 28 for connecting the electroporation electrode 24 with the electroporation electrode-connecting terminal 23; and an adhesive insulator layer 27 which is attached on the skin to insulate such that the conductor 28 or the electroporation electrode 24 does not unnecessarily come into contact with the skin.

The insulin lispro-containing layer 25 is formed by dispersing a thickener in an insulin lispro solution. The insulin lispro release-controlling membrane 29 is not particularly limited, but it is preferable that the membrane does not prevent the permeation of insulin lispro. Thus, a porous membrane having pores is preferably used. For retentivity and permeability of the drug, the pore size of a porous membrane is preferably between 0.01 and 10 μm, and more preferably between 0.1 and 5 μm.

Examples of a material for an insulin lispro release-controlling membrane may include, but are not limited to, nylon membrane, polyvinylidene fluoride, cellulose, nitrocellulose, polycarbonate, polysulfone, polyethylene, nonwovenfabric, gauze, woven cloth, paper, absorbent cotton, porous membranes and foams having communicated pores formed of a material such as polyethylene, polypropylene, vinyl acetate, polyolefin foam, polyamide foam or polyurethane, and products obtained by chemically modifying or treating these materials. Insulin lispro may also be retained in a dry state on the membrane, as describe later.

The insulin lispro-containing layer 25 may comprise an electrolyte, absorbefacient, stabilizer, pH adjuster, thickener, tackiness agent, surfactant, emulsifier, nonwoven fabric, or the like, as well as insulin lispro.

As a material for a backing, any material being excellent in processability, flexibility, and moderate shape retention maybe used. Examples of such a material may include: nonwoven fabric; chlorine-containing resins that are polymers, such as vinylidene chloride, or vinyl chloride; polymers such as olefins, esters, styrenes, acryls, amides, oxymethylenes, phenylene sulfides, amideimides, acrylonitriles, ether ketone, ether sulfone, sulfone, ether imide, butadiene, or isoprene; and copolymers thereof. However, examples are not limited thereto. Products obtained by converting the aforementioned materials into a film state, processed goods thereof, or molded products thereof are used. The thickness of a backing is not particularly limited, but a thickness between 5 and 250 μm is preferable in terms of excellent shape retention and flexibility.

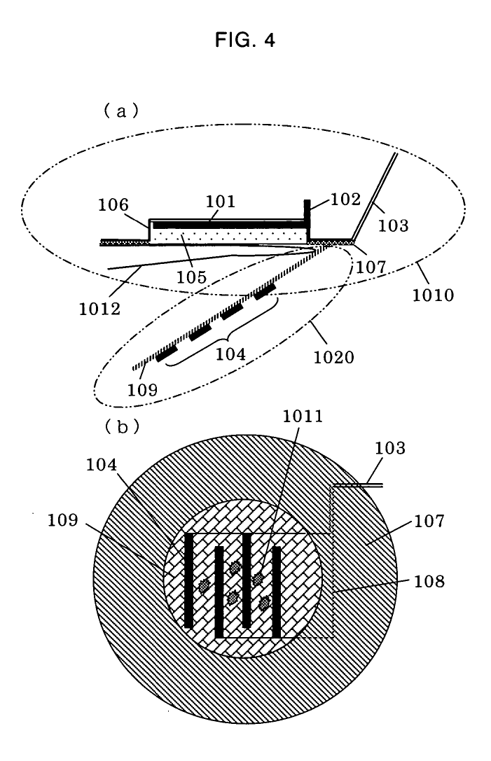

FIG. 4 is a view showing another example of the electroporation-iontophoresis formulation of the present invention. FIG. (a) shows a sectional view thereof, and FIG. (b) shows a plan view thereof. This formulation differs from the formulations shown in FIGS. 2 and 3 in that it has a retaining membrane for retaining insulin lispro in a dry state. This is to say, as shown in the figure, the present formulation is divided by a liner 1012 into: a portion 1010 comprising an iontophoresis electrode and a hydrophilic matrix; and a portion 1020 comprising a dry-state insulin lispro and electroporation electrodes. The portion 1010 comprises a backing 106 having a concave portion, an iontophoresis electrode 101 disposed on the bottom of the concave portion of the backing 106, an iontophoresis electrode-connecting terminal 102 for connecting the iontophoresis electrode 101 with an external power supply, and a hydrophilic matrix base 105 disposed inside the backing 106. The portion 1020 comprises a retaining membrane 109, a dry-state insulin lispro 1011 disposed on the retaining membrane, an electroporation electrode 104 fixed or printed on the retaining membrane, an adhesive insulator layer 107 which covers the peripheral portion of the retaining membrane, a conductor 108 of the electroporation electrode which is protected by the adhesive insulator layer 107, and an electroporation electrode terminal 103 which is connected with the conductor 108.

Herein, before use of the formulation, the insulin lispro 1011 is in a dry powder state on the retaining membrane 109. When it is used, the liner 1012 is pulled out of the portion 1010 comprising the backing 106, the iontophoresis electrode 101, and the hydrophilic matrix base 105, so as to exfoliate it. Thereafter, the insulin lispro-retaining membrane 109 is combined with the portion 1010. Thus, insulin lispro existing on the retaining membrane 109 is dissolved, so that the formulation formed by combining the portion 1010 with the insulin lispro-retaining membrane 109 can adopt a form ready for administration. Since insulin lispro may hardly be dissolved depending on pH, a solubilizer may be added to the dissolved portion thereof. Moreover, measures may be taken against the problem, such that the crystal condition of insulin lispro in the dry state is changed.

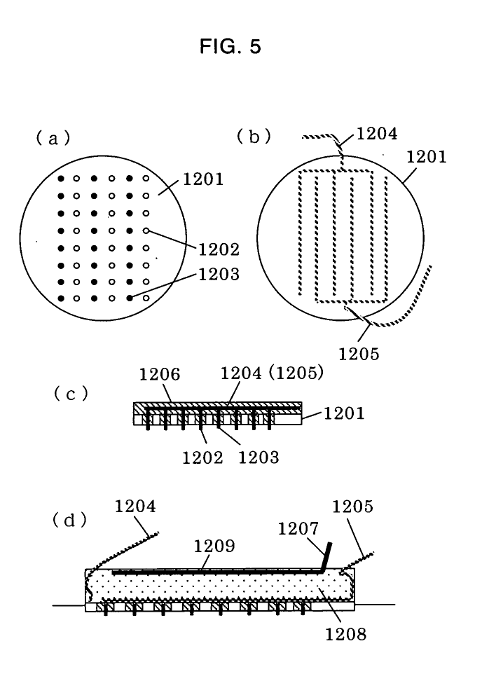

FIG. 5 is a view showing another example of the electroporation-iontophoresis formulation of the present invention. FIG. (a) shows a plan view of an applied plane thereof, FIG. (b) shows a plan view of a conductive layer thereof, FIG. (c) is a partial sectional view of a porous membrane thereof, and FIG. (d) is an overall sectional view thereof. This formulation differs from the formulations shown in FIGS. 2 to 4 in that it has multipoint contact-type electroporation electrodes. This is to say, in the present formulation, multipoint contact-type electroporation electrodes 1202 and 1203 are disposed on a porous membrane 1201, so that they come into contact with the skin in a punctiform. In order that the electrodes are energized through the skin, the periphery of the electrodes is covered with a dielectric layer 1206. Electric current flows through terminals 1204 and 1205, so as to apply voltage during electroporation. As with the periphery of the electrodes, the terminals 1204 and 1205 are also covered with dielectrics, so as to prevent a leakage of current. A formulation 1240 comprising the multipoint contact-type electroporation electrodes has an iontophoresis electrode 1209 as well as the electroporation electrodes. The iontophoresis electrode 1209 is connected with an external power supply via an iontophoresis electrode terminal 1207. In the present formulation, insulin lispro may be contained in a conductive layer 1208, but it may also be contained in the porous membrane 1201.

### EXAMPLES

The present invention will be described in detail in the following examples and Comparative Examples.

In Example 1 and Comparative Examples 1 and 2, a solution containing approximately 500 units of insulin lispro was used. A method of preparing the solution will be described below. A commercially available product Humalog (manufactured by Eli Lilly) was subjected to centrifugal filtration and freeze-drying, so that low molecular weight compounds including ions were eliminated, thereby concentrating the product. Thereafter, the concentrated product was dissolved in a 0.2 N sodium hydroxide aqueous solution, and the obtained solution was neutralized with 0.2 N hydrochloric acid, so as to obtain an insulin lispro solution, which had pH of approximately 7 and a concentration of approximately 500 units/mL.

With regard to a human insulin solution used in Comparative Example 3, a commercially available product Humalin (manufactured by Shionogi & Co., Ltd.) was subjected to the same operations as for insulin lispro, so as to obtain a human insulin solution, which had pH of approximately 7 and a concentration of approximately 500 units/mL.

With regard to insulin solutions used in Example 2 and Comparative Example 4, a commercially available 100 units/mL Humalog and a commercially available 100 units/mL Humalin were directly used, respectively.

Further, in Comparative Examples 5 to 7, various types of insulin were adjusted to have a concentration of 100 units/mL, and were used.

### Example 1

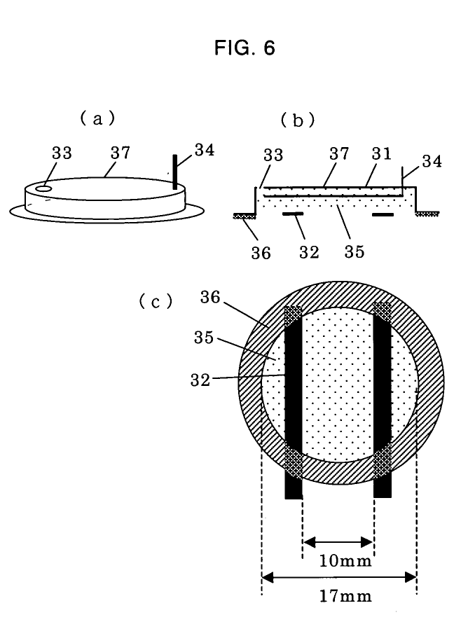

FIG. 6 is a view showing the electroporation-iontophoresis formulation used in the present examples. FIG. (a) is a perspective view thereof, FIG. (b) is a sectional view thereof, and FIG. (c) is a plan view thereof. As shown in the figure, the present formulation comprises: a backing 37 having a concave portion; an iontophoresis electrode 31 disposed on the bottom of the concave portion of the backing 37; an iontophoresis electrode-connecting terminal 34 for connecting the iontophoresis electrode 31 with an external power supply; an insulin lispro aqueous solution layer 35 disposed inside the backing 37; a pair of electroporation electrodes 32 disposed on the insulin lispro aqueous solution layer 35; an adhesive insulator layer 36 which is attached on the skin to insulate such that the electroporation electrodes do not unnecessarily come into contact with the skin; and a port 33 for supplying the insulin lispro solution.

Herein, the concave portion of the backing 37 has a circular section with a diameter of 17 mm. The electroporation electrodes 32 for applying high voltage were made by silver foil, and the electrodes were disposed such that a distance between them was 10 mm. A silver/silver chloride electrode was prepared by electrolysis of silver foil, and it was used as the iontophoresis electrode 31 for applying low voltage, which was located on the side containing insulin lispro. The iontophoresis electrode 31 was connected with an iontophoresis power supply via the iontophoresis electrode-connecting terminal 34. An insulin-administering device containing the present formulation was attached to the abdominal region of SD rats. Thereafter, an insulin lispro solution (approximately 500 U/mL) containing the present formulation was supplied through the port 33, and it was defined as the insulin lispro aqueous solution layer 35. At that time, the iontophoresis electrode was located in the solution. Using the electroporation 32, a voltage of 150 V was applied 10 times at a pulse width of 10 milliseconds. Thereafter, the iontophoresis electrode 31 was connected with the iontophoresis power supply via the iontophoresis electrode-connecting terminal 34, and a direct current of 0.31 mA was applied for 1 hour. The blood was collected from the carotid artery of the rats over time. Using an insulin lispro measurement kit (Insulin Lispro RIA Kit; manufactured by Linco Research) and a glucose measurement kit (Glucose CII Test Wako; manufactured by Wako Pure Chemical Industries, Ltd.), the level of insulin lispro in the blood and the level of glucose therein were measured.

### Example 2

An experiment was carried out in the same manner as in Example 1 with the exception that the unit of the insulin lispro solution to be administered was set at 100 units/mL.

### Comparative Example 1

### The Case of Using Only the Iontophoresis

A device formed by removing the electroporation electrodes 32 from the device shown in FIG. 6 was used. The insulin lispro solution was administered, and the iontophoresis electrode 31 was connected with an iontophoresis power supply via the iontophoresis electrode-connecting terminal 34. Thereafter, a direct current of 0.31 mA was applied for 1 hour. An insulin lispro level and a glucose level in the blood were measured in the same manner as in Example 1.

### Comparative Example 2: The Case of Using Only the Electroporation)

A device formed by removing the iontophoresis electrode 31 from the device shown in FIG. 6 was used. The insulin lispro solution was administered. Thereafter, using the electroporation electrodes 32, a voltage of 150 V was applied 10 times at a pulse width of 10 milliseconds. An insulin lispro level and a glucose level in the blood were measured in the same manner as in Example 1.

### Comparative Example 3

### A Case of Using Human Insulin

An experiment was carried out in the same manner as in Example 1 with the exception that a human insulin solution (500 units/mL) was used instead of insulin lispro.

The insulin level was measured using a blood insulin measurement kit (IMX Insulin Dainapack; manufactured by Dainabot). In addition, the glucose level was measured with Glucose CII Test Wako, as in the case of Example 1.

### Comparative Example 4

### Another Case of Using Human Insulin

An experiment was carried out in the same manner as in Example 1 with the exception that Humalin (100 units/mL; manufactured by Shionogi & Co., Ltd.) was used as human insulin instead of insulin lispro.

The insulin level was measured using a blood insulin measurement kit (IMX Insulin Dainapack; manufactured by Dainabot). In addition, the glucose level was measured with Glucose CII Test Wako, as in the case of Example 1.

### Comparative Example 5

### The Case of Using Swine Insulin

An experiment was carried out in the same manner as in Example 1 with the exception that swine insulin (100 U/mL; manufactured by Sigma) was used instead of insulin lispro.

The level of swine insulin in the blood was not measured, but only the level of glucose in the blood was measured.

### Comparative Example 6

### The Case of Using Bovine Insulin

An experiment was carried out in the same manner as in Example 1 with the exception that bovine insulin (100 U/mL; manufactured by Sigma) was used instead of insulin lispro.

The level of bovine insulin in the blood was not measured, but only the level of glucose in the blood was measured.

### Comparative Example 7

### The Case of Using Arginine-Insulin

An experiment was carried out in the same manner as in Example 1 with the exception that arginine-insulin (100 U/mL; manufactured by Sigma) was used instead of insulin lispro.

The level of insulin in the blood was not measured, but only the level of glucose in the blood was measured.

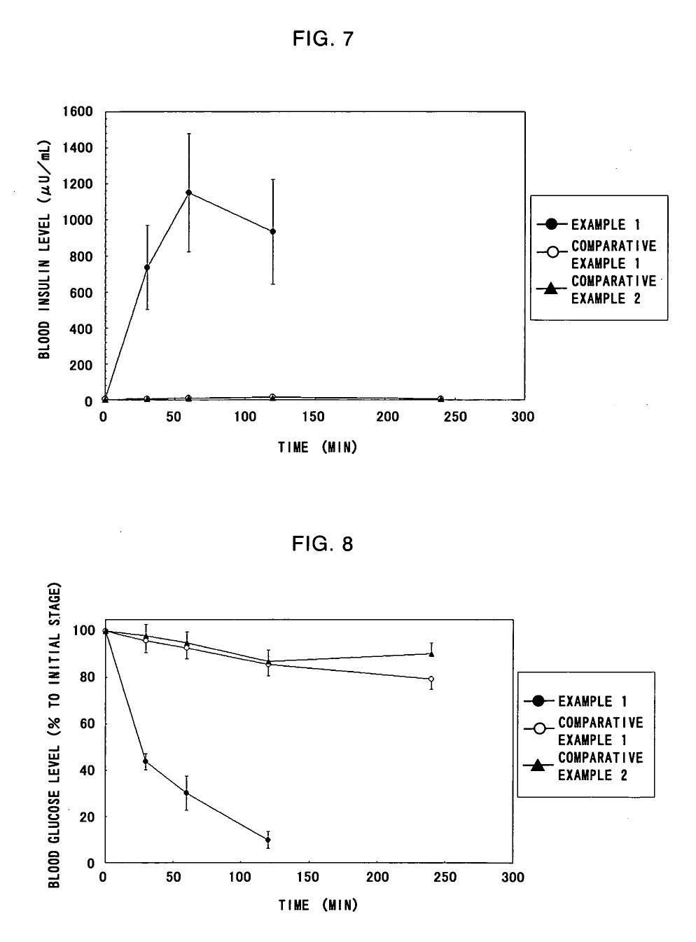

FIG. 7 is a graph showing a comparison made among the insulin lispro levels in the blood, in Example 1 and Comparative Examples 1 and 2. The iontophoresis and electroporation of the present invention were applied in Example 1, only the iontophoresis was applied in Comparative Example 1, and only the electroporation was applied in Comparative Example 2. Insulin lispro was administered in all the examples.

As is clear from FIG. 7, a blood insulin lispro level of 1200 μU/mL was observed at maximum in Example 1, but almost no insulin lispro was detected in both Comparative Examples 1 and 2.

FIG. 8 is a graph showing a change in the level of glucose in the blood in Example 1 and Comparative Examples 1 and 2, as a ratio of the blood glucose level after administration to the blood glucose level at the initial stage (before administration).

In Example 1, the blood glucose level was decreased to 9% of the initial value only at 120 minutes after administration. Thereafter, high absorption of insulin lispro was observed to such an extent that several rats died due to hypoglycemia. In contrast, in Comparative Examples 1 and 2, a decrease in the blood glucose level was only approximately 80% of the initial value at 120 minutes after administration. This is to say, the results show that only the combined use of the electroporation and the iontophoresis enables high absorption of insulin lispro.

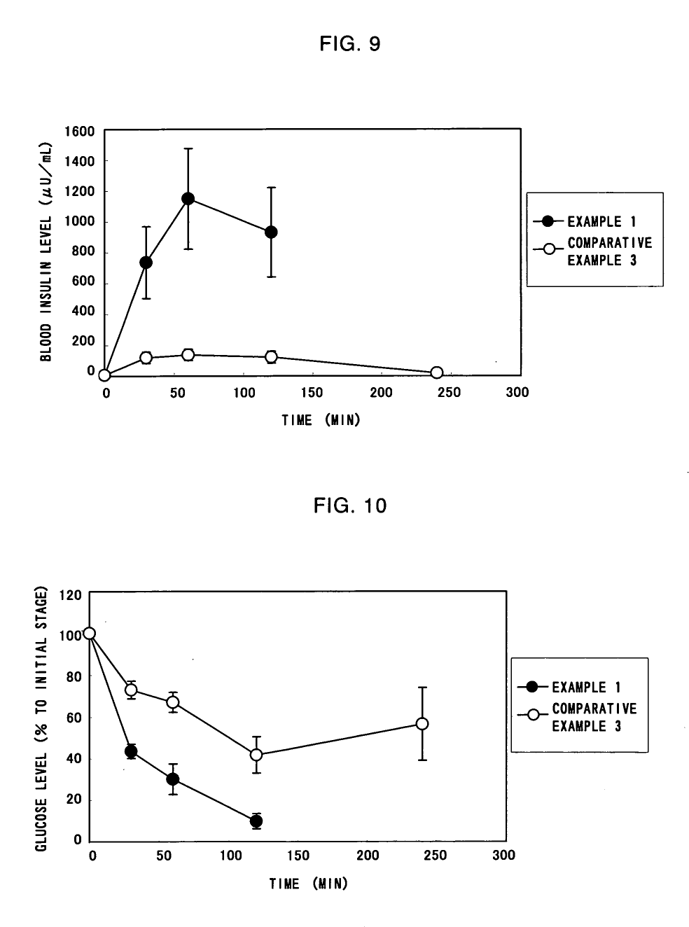

FIG. 9 is a graph showing the level of insulin in the blood in Example 1 and Comparative Example 3. FIG. 9 shows a comparison regarding the absorption of other types of insulin in the case of using the electroporation and the iontophoresis. The figure specifically shows the level of insulin in the blood in Example 1 (wherein insulin lispro was administered by using the electroporation and the iontophoresis) and that in Comparative Example 3 (wherein human insulin was administered by using the electroporation and the iontophoresis). In both examples, the type of insulin used was different, but the dosage (approximately 500 U/mL) was the same. As with the results shown above, a blood insulin level of 1200 μU/mL was observed at maximum in Example 1, but only a blood insulin level of 100 μU/mL was observed in Comparative Example 3.

FIG. 10 is a graph showing a change in the level of glucose in the blood in Example 1 and Comparative Example 3, as a ratio of the blood glucose level after administration to the blood glucose level at the initial stage (before administration). As shown in FIG. 10, with regard to beneficial effects also, insulin lispro was decreased to 9% of the initial value at 120 minutes after administration, but human insulin was decreased to only approximately 40% of the initial value. This is to say, from the results of the comparison made between Example 1 and Comparison example 3, it is found that although the electroporation and the iontophoresis are used in combination, sufficient absorption cannot be achieved unless a drug to be administered is insulin lispro.

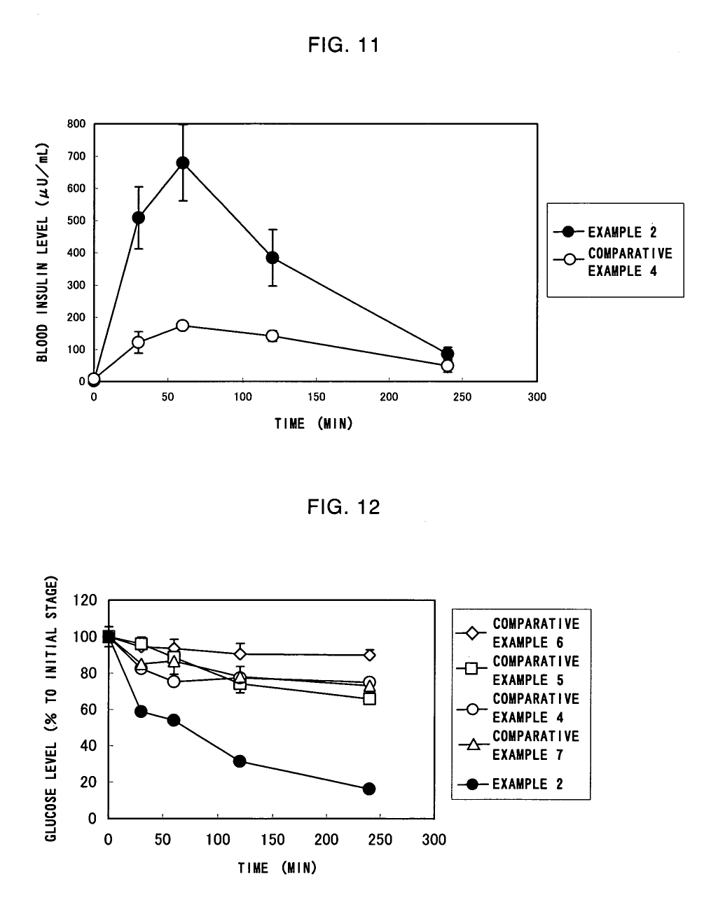

FIG. 11 is a graph showing the level of insulin in the blood in Example 2 and Comparative Example 4. Namely, FIG. 11 shows the blood insulin level obtained in a case where the concentration of each of an insulin lispro solution and a human insulin solution was set at 100 U/mL and where such insulin was administered using the electroporation and the iontophoresis in combination.

Even though the concentration of insulin administered in Example 2 was set at one-fifth of the concentration in Example 1, the maximum blood insulin level was approximately 700 μU/mL. Thus, when compared with Comparative Example 4, extremely high absorption of insulin was achieved in Example 2.

FIG. 12 is a graph showing a change in the level of glucose in the blood in Example 2 and Comparative Examples 4, 5, 6, and 7, as a ratio of the blood glucose level after administration to the blood glucose level at the initial stage (before administration). This is to say, FIG. 12 shows a change in the level of glucose in the blood over time, when other types of insulin (arginine-insulin, swine insulin, and bovine insulin) were administered in a concentration 100 U/mL that was the same as in Example 2 and Comparative Example 4, using the electroporation and the iontophoresis in combination. As is clear from FIG. 11, only in the case of using insulin lispro, the glucose level was decreased to 20% of the initial value, but when other types of insulin were used, a decrease in the glucose level was only 65% of the initial value.

From the experimental examples shown herein, it was confirmed that extremely high absorption of insulin can be achieved only when insulin lispro is administered by using in combination the electroporation capable of applying a high electric field for a very short time and the iontophoresis capable of applying a low electric field for a long time. Also, high beneficial effects were confirmed in this case. When either the iontophoresis or the electroporation was used, no effects could be obtained. Although both means were used in combination, absorption was insufficient in the case of administering other types of insulin other than insulin lispro.

That is to say, the present invention was made based on the findings that the combined use of electroporation and iontophoresis enables significantly high absorption of insulin when it is administered percutaneously or transmucosally.

Several examples of prescription used for the above described electroporation-iontophoresis formulation are shown below as Examples 3 to 7. Compositions prepared according to such prescription may be applied to the above described electroporation-iontophoresis formulation, so as to use as a formulation to be administered.

### Example 3

| | |
|---|---|
| Insulin lispro (500 U solution) | 0.2 mL |
| 15% polyvinyl alcohol aqueous gel | 1 g |

### Example 4

| | |
|---|---|
| Insulin lispro (500 U solution) | 0.2 mL |
| Carboxymethylcellulose sodium | 30 mg |
| Water | 0.77 g |

### Example 5

| | |
|---|---|
| Insulin lispro (500 U solution) | 0.2 mL |
| Agar | 10 mg |
| locust bean gum | 3 mg |
| Water | 0.787 g |

### Example 6

| | |
|---|---|
| Insulin lispro (500 U solution) | 0.2 mL |
| Xanthan gum | 3 mg |
| locust bean gum | 3 mg |
| Water | 0.787 g |

### Example 7

| | |
|---|---|
| Insulin lispro (500 U solution) | 0.2 mL |
| Polyacrylic acid | 50 mg |
| Aluminum hydroxide | 5 mg |
| Water | 0.745 g |

### INDUSTRIAL APPLICABILITY

According to the present invention, an insulin-administering device enabling the effective administration of insulin via a percutaneous or submucous administration route can be obtained.

1. An insulin-administering device for percutaneously or transmucosally administering insulin lispro represented by the structural formula indicated below or a pharmaceutically acceptable salt thereof (hereinafter referred to as “insulin lispro”), using at least two different electric field-applying means.

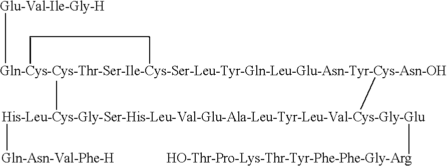

2. The insulin-administering device according to claim 1, wherein the two different electric field-applying means are iontophoresis and electroporation.

3. The insulin-administering device according to claim 2, wherein the electric current applied during iontophoresis is between 0.01 and 1.0 mA/cm².

4. The insulin-administering device according to claim 2, wherein the voltage applied during electroporation is between 1 V/cm and 10 kV/cm.

5. The insulin-administering device according to claim 1, wherein said insulin lispro is dissolved, suspended, or dispersed in a hydrophilic matrix.

6. The insulin-administering device according to claim 5, wherein the hydrophilic matrix comprises one or more selected from the group consisting of agar, locust bean gum, xanthan gum, polyvinyl alcohols and derivatives thereof, and polyacrylic acid and salts thereof.

7. The insulin-administering device according to claim 1, wherein said device comprises a membrane for controlling the release of said least one of the insulin lispros.

8. The insulin-administering device according to claim 7, wherein at least a pair of electrodes used for electroporation is disposed on the release-controlling membrane.

9. The insulin-administering device according to claim 7, wherein the release-controlling membrane is formed of a porous membrane.

10. The insulin-administering device according to claim 1, wherein said insulin lispro is retained on the membrane.

11. The insulin-administering device according to claim 10, wherein said insulin lispro is retained in a dry state on the membrane and in that a part or all of said insulin lispro is dissolved when it is used.

12. The insulin-administering device according to claim 2, wherein at least one of the electrodes used for electroporation is disposed directly on the skin or mucosa, or adjacent thereto.

13. An insulin-administering device, wherein said device comprises an electroporation-iontophoresis formulation containing insulin lispro, a reference formulation that is a counter electrode in iontophoresis, and a power supply connected to both formulations.

14. The insulin-administering device according to claim 13, wherein the power supply has a connecting port used for iontophoresis and a connecting port used for electroporation.

15. An electroporation-iontophoresis formulation, wherein said formulation comprises a backing, an iontophoresis electrode disposed on the backing, an insulin lispro-containing layer which is disposed on the iontophoresis electrode and contains an insulin lispro, and electroporation electrodes which are disposed on the insulin lispro-containing layer and have polarities different from one another.

16. The electroporation-iontophoresis formulation according to claim 15, wherein a release-controlling membrane for controlling the release of said insulin lispro is provided between the insulin lispro-containing layer and the electroporation electrodes.

17. The electroporation-iontophoresis formulation according to claim 16, wherein the release-controlling membrane is a porous membrane having a pore size between 0.01 and 10 μm.

18. An electroporation-iontophoresis formulation, wherein said formulation comprises a backing, an iontophoresis electrode disposed on the backing, a hydrophilic matrix base disposed on the iontophoresis electrode, a liner disposed on the hydrophilic matrix base, a retaining membrane which is disposed on the liner and retains an insulin lispro, and electroporation electrodes which are disposed on the retaining membrane and have polarities different from one another.

19. The electroporation-iontophoresis formulation according to claim 18, wherein said insulin lispro is retained in a dry state on the retaining membrane.

20. The electroporation-iontophoresis formulation according to claim 15, wherein the electroporation electrodes are formed as a multipoint contact-type.
